(19) United States
(12) Patent Application Publication
(10) Pub. No.: US 2007/0142884 A1
(43) Pub. Date: Jun. 21, 2007

(54) Methods and apparatuses for treating an esophageal disorder such as gastroesophageal reflux disease

(75) Inventors: Sally D. Jandrall, Seattle, WA (US); William S. Helton, Glencoe, IL (US)

Correspondence Address: PERKINS COIE LLP;PATENT-SEA, P.O. BOX 1247, SEATTLE, WA, 98111-1247, US

(73) Assignee: AcousTx Corporation, Seattle, WA (US)

(21) Appl. No.: 11/303,146

(22) Filed: Dec. 16, 2005

(51) Int. Cl. A61F 7/00; A61F 7/12

(52) U.S. Cl. 607/96

(57) ABSTRACT

Methods and apparatuses for treating esophageal disorders such as gastroesophageal reflux disease are disclosed herein. One embodiment of a method includes applying energy to a portion of the sling and/or clasp fibers at the stomach, gastroesophageal junction, and/or esophagus of the patient in a manner that shortens or otherwise alters the fibers. The altered sling and/or clasp fibers are expected to recalibrate and restore the cardia and improve the competence of the lower esophageal sphincter. The energy applied to the fibers can be ultrasonic, radio-frequency, microwave, light, and/or other suitable types of energy.

### TECHNICAL FIELD

The present invention is related to methods and apparatuses for treating an esophageal disorder such as gastroesophageal reflux disease.

### BACKGROUND

Gastroesophageal reflux disease (GERD) is a common gastroesophageal disorder in which the stomach contents reflux into the lower esophagus due, in part, to a dysfunction of the lower esophageal sphincter (LES). The antireflux barrier in normal individuals is a highly competent structure that withstands enormous pressures without allowing reflux. For example, a 250-lb wrestler can land on his opponent's abdomen without causing the opponent to vomit. The LES maintains a resting pressure higher than the pressure in the adjacent esophagus or stomach. This high pressure zone separates the gastric cavity from the esophageal lumen. Stomach contents are usually acidic. Hence, gastric reflux into the lower esophagus due to LES dysfunction is potentially injurious to the esophagus resulting in a number of possible complications of varying medical severity. The reported incident of GERD in the U.S. is as high as 10% of the population.

Acute symptoms of GERD include heartburn, laryngeal problems, pulmonary disorders and chest pain. On a chronic basis, GERD subjects the esophagus to ulceration and inflammation, and may result in more severe complications including esophageal obstruction, acute and/or chronic blood loss, and cancer. In fact, the increasing incidence of adenocarcinoma of the esophagus, which is rising faster than any other cancer, is believed to be directly linked to the increasing incidence and severity of GERD. GERD typically requires lifelong medical therapy or surgery for the management of patients with frequent symptoms.

Current drug therapy for GERD includes proton-pump inhibitors (PPI) that reduce stomach acid secretion and other drugs which may completely block stomach acid production. However, while pharmacologic agents often provide symptomatic relief and allow esophagitis to heal, they do not address the underlying cause of LES dysfunction. Drug therapy is also expensive, and may impair digestion.

A number of invasive procedures have been developed in an effort to correct the dysfunctional LES in patients with GERD. The role of surgery is to restore the function of the incompetent antireflux barrier. One such procedure, gastric fundoplication, involves wrapping the gastric fundus, partially or completely around the lower esophagus. This anatomic rearrangement results in the creation of an increased zone of high intragastric pressure following meals that can prevent reflux of gastric contents into the esophagus. However, the gastroesophageal junction is more than a flaccid rubber tube; in order for a gastric fundoplication to be effective, it must restore several aspects of the dysfunctional anatomy and physiology that exists in patients with GERD. First, in those with a hiatal hernia in which the LES has moved above the diaphragmatic hiatus into the chest where pressure is less than the abdomen, the operation must restore the position of the GE junction and LES below the diaphragm. Second, the esophageal crura must be approximated and the GE junction secured below the diaphragm to prevent recurrent herniation and migration of the LES above the diaphragm again. Thirdly, the fundoplication must also produce a recalibration of the cardia. Calibration of the cardia narrows the angle of His and improves the coincidence of the mucosal seal and the size of the mucosal contact zone. Classic antireflux surgery does not, however, always restore all of these aspects of the dysfunctional anatomy, which could explain why antireflux surgery fails in a significant number of patients, especially those with long-segment and complicated Barrett's esophagus. Although gastric fundoplication has a high rate of success, it is an open abdominal procedure with the usual risks of abdominal surgery including: postoperative infection, herniation at the operative site, internal hemorrhage, and perforation of the esophagus or the cardia.

Recently, gastric fundoplication has been able to be performed using minimally invasive surgical techniques. This procedure involves essentially the same steps as an open gastric fundoplication with the exception that surgical manipulation is performed through several small incisions by way of surgical trocars inserted at various positions in the abdomen. This less invasive surgical approach is capable of restoring the LES similar to the open operation but patients recover from surgery quicker and with less discomfort.

As an alternative to open or minimally invasive surgery, a number of endoluminal techniques have been recently developed as treatment options for GERD. These techniques are even less invasive than the laparoscopic gastric fundoplication in that devices are inserted through the mouth into the esophagus to reach the area of the LES. One such technique, disclosed in U.S. Pat. No. 5,088,979, uses an invagination device containing a number of wires and needles which are in a retracted position inserted transorally into the esophagus. Once positioned at the LES, the needles are extended to engage the esophagus and fold the attached esophagus beyond the gastroesophageal junction. A remotely operated stapling device, introduced percutaneously through an operating channel in the stomach wall, is actuated to fasten the invaginated gastroesophageal junction to the surrounding involuted stomach wall.

Another device is disclosed in U.S. Pat. No. 5,676,674. In this procedure, invagination is performed with a jaw-like device, and the invaginated gastroesophageal junction is fastened to the fundus of the stomach with a transoral approach using a remotely operated fastening device, eliminating the need for an abdominal incision. However, this procedure is still traumatic to the LES and presents the post-operative risks of gastroesophageal leaks, infection, and foreign body reaction, the latter sequela resulting when foreign materials such as surgical staples are implanted in the body.

Curon Medical has developed a radio-frequency ablation device (disclosed in U.S. Pat. No. 6,846,312) that is also delivered to the gastroesophageal junction transorally. The device first penetrates the esophagus with RF electrodes arranged in a circular fashion. RF energy is delivered into the muscular tissues to cause a tightening of the LES through the generation of lesions in the tissue. There have been a number of major complications resulting from this device, and its effectiveness is debated.

There are also several device approaches based on the idea of injecting bulking agents into the LES. They suffer from short-term effectiveness. Enteryx (now owned by Boston Scientific Corp.) is the only FDA approved device based on this approach. Each injection of the implanted material is performed with the aid of fluoroscopy to ensure accurate deep mural placement of the implant. Concomitant endoscopic imaging is utilized to avoid misdirected large volume submucosal implants, which will ulcerate the esophageal mucosa and slough off if not placed deep within the muscle.

### BRIEF DESCRIPTION OF THE DRAWINGS

FIG. 1 is a schematic representation of a portion of the human anatomy including an esophagus, a stomach, and a gastroesophageal junction (or cardia).

FIG. 2 is a schematic cross-sectional view of a gastroesophageal junction taken generally along the line A-A of FIG. 1 in an individual with a normal cardia.

FIGS. 3A-3D are schematic representations of the expected orientation and operation of sling and clasp fibers in an individual with a normal cardia.

FIG. 4 is a schematic cross-sectional view of a gastroesophageal junction taken generally along the line A-A of FIG. 1 in an individual with a dilated cardia.

FIGS. 5A-5D are schematic representations of the expected orientation and operation of sling and clasp fibers in an individual with a dilated cardia.

FIG. 6 is a schematic representation of an endoscope inserted into the stomach of a patient in accordance with one embodiment of the invention.

FIG. 7 is a schematic representation of an endoscope positioned in the cardia of the stomach of a patient in accordance with another embodiment of the invention.

FIG. 8 illustrates one example of a pattern of heat affected zones in accordance with one embodiment of the invention.

FIG. 9 is a schematic representation of an endoscope in accordance with another embodiment of the invention.

FIG. 10 is a schematic representation of an endoscope in accordance with another embodiment of the invention.

FIG. 11 illustrates one example of a pattern of welds formed in a patient in accordance with one embodiment of the invention.

### DETAILED DESCRIPTION

A. Overview

The present invention is directed toward methods and apparatuses for treating esophageal disorders such as gastroesophageal reflux disease. One embodiment of a method includes applying energy to a portion of the sling and/or clasp fibers at the stomach, cardia, and/or esophagus of the patient in a manner that shortens at least some of the fibers and thus improves their length-tension properties. The shortened sling and/or clasp fibers are expected to recalibrate and restore the cardia and improve the competence of the LES. The energy applied to the sling fibers and/or clasp fibers can be ultrasonic, radio-frequency, microwave, light, and/or other suitable type of energy.

In another embodiment, a method includes inserting a probe into the patient, and transmitting energy from the probe toward a portion of the sling and/or clasp fibers at the stomach and/or cardia of the patient to form welds in some of the individual fibers. The welds in the fibers shorten the length of the fibers such that the competence of the LES is restored or at least improved.

Another aspect of the invention is directed to apparatuses for treating esophageal disorders such as gastroesophageal reflux disease. In one embodiment, an apparatus includes an endoscope for insertion into the patient and a securing device coupled to the endoscope. The securing device is configured to reliably secure a portion of tissue containing sections of sling fibers and/or clasp fibers at the stomach and/or gastroesophageal junction of the patient. The apparatus further includes a applicator coupled to the endoscope and positioned for applying energy to the portion of tissue containing sections of sling fibers and/or clasp fibers secured by the securing device.

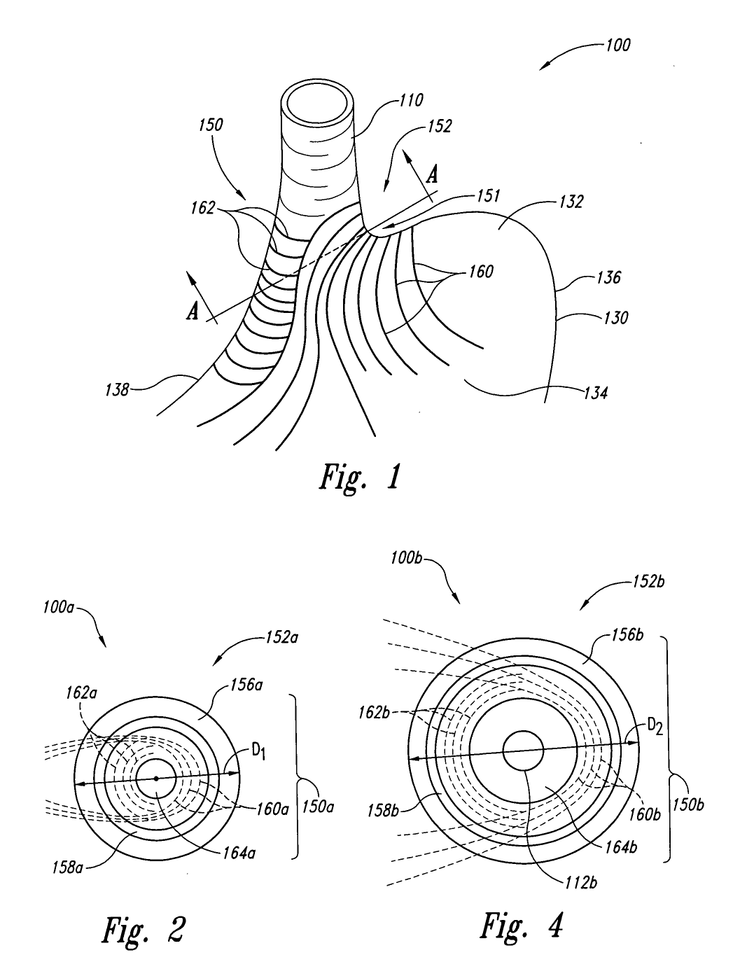

The following disclosure describes methods and apparatuses for treating esophageal disorders such as gastroesophageal reflux disease in patients. Unless the term “or” is expressly limited to mean only a single item exclusive from the other items in reference to a list of two or more items, then the use of “or” in such a list is to be interpreted as including (a) any single item in the list, (b) all of the items in the list, or (c) any combination of items in the list. Certain details are set forth in the following description and in FIGS. 1-11 to provide a thorough understanding of various embodiments of the invention. Other details describing the operation, anatomy, and physiology of portions of the gastrointestinal tract are not set forth in the following disclosure to avoid unnecessarily obscuring the description of various embodiments of the invention.

Many of the details, positions, and other features shown in the figures are merely illustrative of particular embodiments of the invention. Accordingly, other embodiments can have other details, positions, and/or features without departing from the spirit or scope of the present invention. In addition, further embodiments of the invention may be practiced without several of the details described below, or various aspects of any of the embodiments described below can be combined in different combinations.

B. Gastrointestinal Tract and Gastroesophageal Reflux Disease

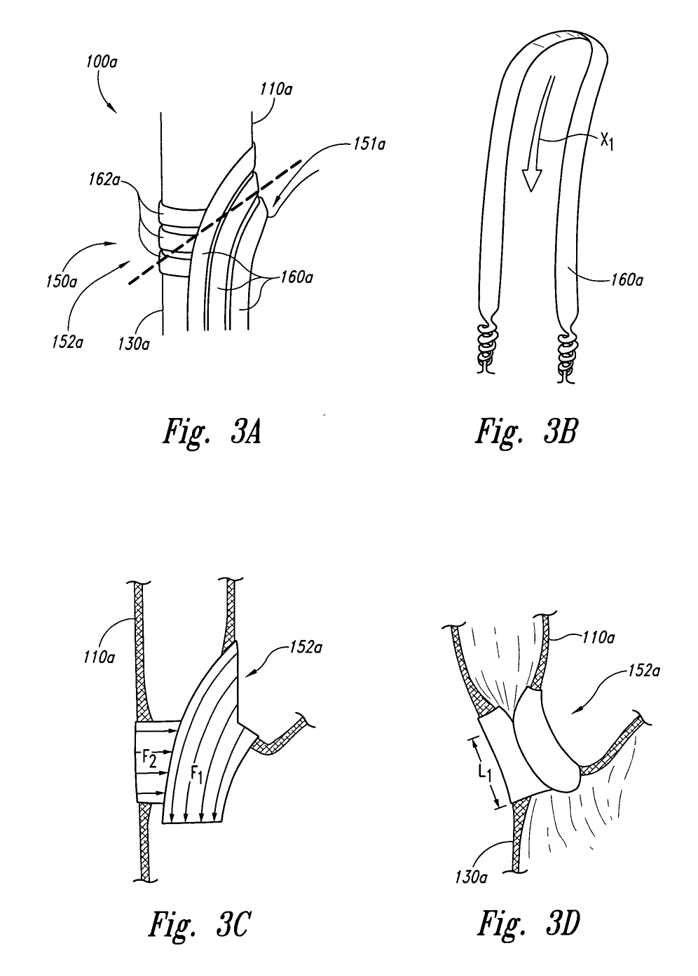

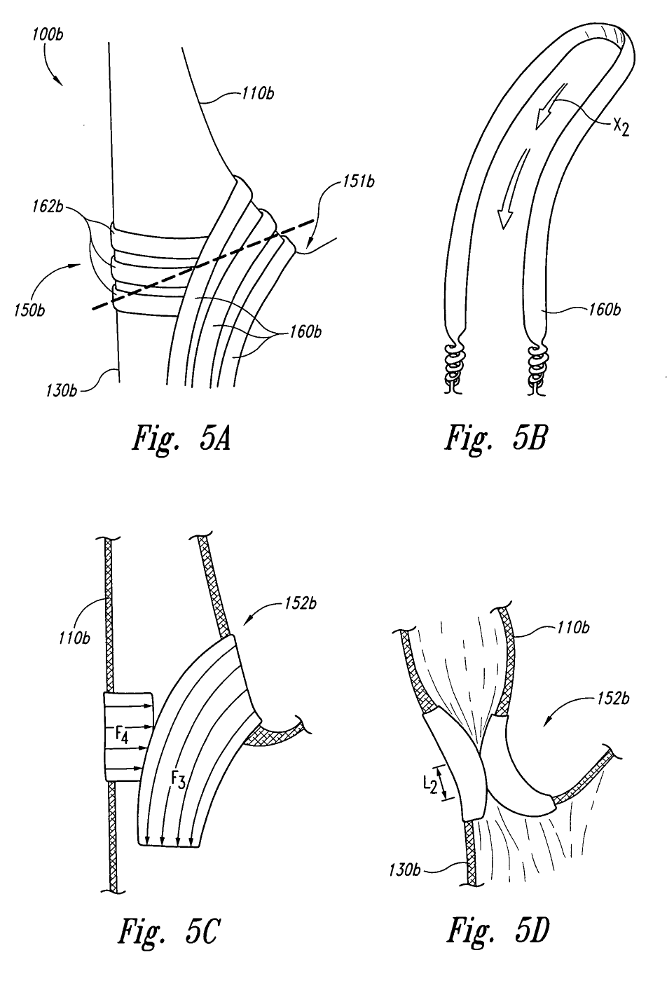

FIG. 1 is a schematic representation of an internal portion of an individual 100 including an esophagus 110, a stomach 130, and a gastroesophageal junction (or cardia) 150 between the esophagus 110 and the stomach 130. The terms gastroesophageal junction and cardia are used interchangeably herein. The stomach 130 has a fundus 132 adjacent to the cardia 150, a body 134 adjacent to the fundus 132, a greater curvature 136 extending around the body 134 and a portion of the fundus 132, and a lesser curvature 138 extending around the body 134 and ending at the gastroesophageal junction 150. The gastroesophageal junction 150 has an angle of “His” 151 between the esophagus 110 and the stomach 130 and a lower esophageal sphincter 152 at the end of the esophagus 110. The lower esophageal sphincter 152 is comprised of two muscular groups, namely gastric sling fibers 160 (shown in the figures as lines) and semicircular clasp fibers 162 (shown in the figures as lines). The sling fibers 160 have a generally oblique orientation and extend from the body 134 of the stomach 130 over the angle of His 151 to form a sling-like structure. The clasp fibers 162 are generally semicircular fibers positioned generally transverse to the sling fibers 160. The sling and clasp fibers 160 and 162 operate together to form the lower esophageal sphincter 152 and maintain the high pressure zone that confines the gastric environment to the stomach. The operation of the sling and clasp fibers 160 and 162 is described in greater detail below with reference to FIGS. 3A-3D and 5A-5D.

The lower esophageal sphincter (LES) 152 selectively inhibits gastric acid and other stomach contents from passing into the lower esophagus 110. In some people, however, the LES 152 becomes mechanically incompetent or dysfunctional, resulting in Gastroesophageal Reflux Disease (GERD). A dysfunctional LES 152 occurs when there is a decrease in LES pressure, the coincidence of the mucosal seal is degraded, and the length of the high pressure zone shortens. There is a correlation between individuals with a dilated cardia 150 (or enlarged perimeter of the gastroesophageal junction) and the severity of GERD. Anatomic dilation of the cardia 152 implies a permanent morphologic change in the gastroesophageal junction, provoked of necessity by an alteration in the architecture or arrangement of the muscular components that shape it. For example, chronic dilation of the cardia 150 alters the function of the sling and clasp fibers 160 and 162. Specifically, dilation of the cardia 150 implies elongation of the sling and clasp muscular fibers 160 and 162, and alteration in their relative angulation and arrangement. The length-tension properties of the elongated muscle fibers are degraded, resulting in reduced LES pressure. Moreover, because of the altered orientation of the sling and clasp fibers 160 and 162, the fibers 160 and 162 may not effectively interact, which also reduces the LES pressure. In addition, alteration of the relative orientation of the sling and clasp fibers 160 and 162 reduces the contact area (the mucosal seal) and shortens the high pressure zone such that the LES 152 is easier to open. Furthermore, the enlarged perimeter of the gastroesophageal junction 150 effectively reduces the LES pressure because less force is required to open the larger diameter (Law of La Place). Moreover, the angle of His 151 may also be increased. Thus, the closing pressure is impaired, and a mechanically defective LES 152 results.

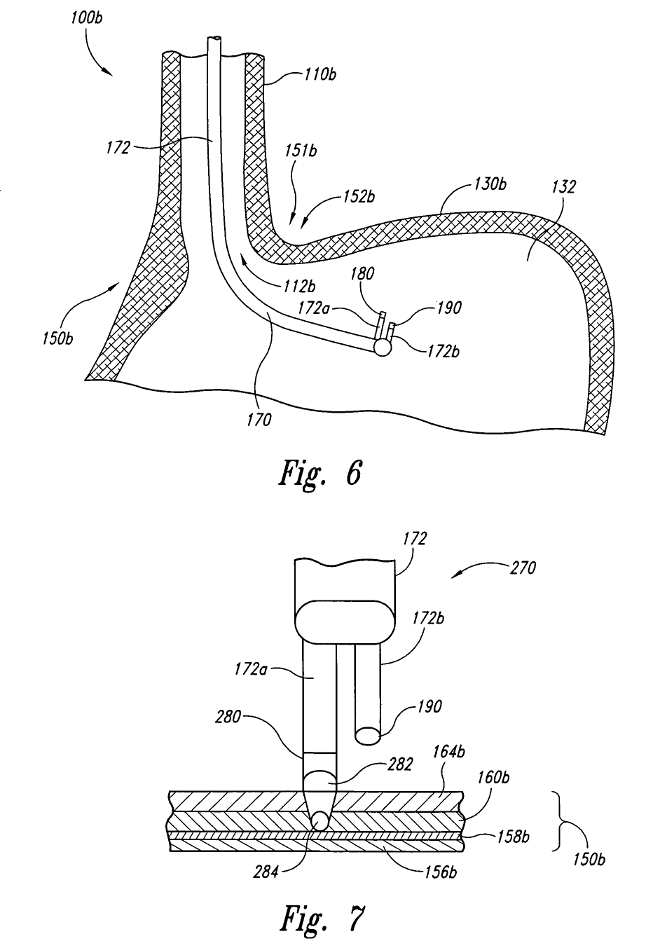

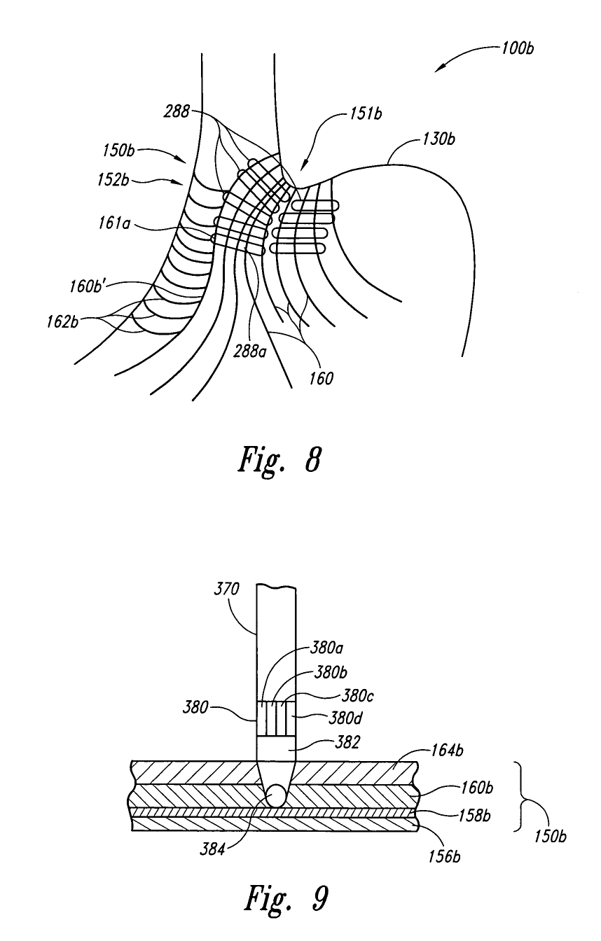

Although a dilated cardia 150 is not the origin of GERD, it represents a point at which the LES 152 becomes mechanically incompetent. Shortening the sling and/or clasp fibers 160 and/or 162 reduces the perimeter of the cardia 150. By the Law of La Place, a reduced perimeter effectively increases the LES pressure. Reduction in the perimeter of the cardia 150 should also recalibrate the cardia 150 by narrowing the angle of His 151, and bring the sling and clasp fibers 160 and 162 back into normal alignment (restoring their force vectors) so that they can function properly together. Restoring the normal alignment of the sling and clasp fibers 160 and 162 increases (a) the LES pressure, (b) the coincidence of the mucosal contact area, and (c) the length of the high pressure zone. Moreover, shortening the sling and clasp fibers 160 and 162 is expected to improve their length-tension properties, which also increases the LES pressure. Therefore, the above-described alterations improve the mechanical function or competence of the LES 152. Several methods and apparatuses for shortening the sling and/or clasp fibers 160 and/or 162 are discussed in detail below with regard to FIGS. 6-11.

FIG. 2 is a schematic cross-sectional view of a gastroesophageal junction 150a taken generally along the line A-A of FIG. 1 in an individual 100a with a normal cardia 150a. The gastroesophageal junction 150a includes a plurality of outer layers 156a, a longitudinal muscle layer 158a radially inward of the outer layers 156a, a plurality of sling and clasp fibers 160a and 162a radially inward of the longitudinal muscle layer 158a, and a mucosa/submucosa layer 164a radially inward of the sling and clasp fibers 160a and 162a. In the normal cardia 150a, the sling and clasp fibers 160a and 162a have normal length-tension properties and are properly positioned relative to each other for effectively operating together and forming a competent LES 152a with a zone of high pressure of normal length and pressure. The normal cardia 150a has a diameter D₁ of approximately 2 centimeters in a healthy adult.

FIGS. 3A-3D are schematic representations of the expected orientation and operation of the sling and clasp fibers 160a and 162a in the individual 100a with the normal cardia 150a. For example, FIG. 3A illustrates the normal orientation of sling and clasp fibers 160a and 162a at the gastroesophageal junction 150a. Specifically, the sling fibers 160a have an oblique orientation and extend from one side of the stomach 130a, over the angle of His 151a, to the other side of the stomach 130a. The clasp fibers 162a have a lateral orientation and a semicircular configuration such that they do not extend completely around the gastroesophageal junction 150a. The sling fibers 160a are positioned generally on one side of the gastroesophageal junction 150a, and the clasp fibers 162a are positioned generally on the other side of the gastroesophageal junction 150a such that the fibers 160a and 162a cooperate to form a competent LES 152a.

FIG. 3B illustrates a force vector X₁ representing the force exerted by the individual sling fibers 160a. The force vector X₁ of the individual sling fibers 160a has a generally vertical orientation. FIG. 3C illustrates a combined force F₁ exerted by the individual sling fibers 160a across a first displacement area and a combined force F₂ exerted by the individual clasp fibers 162a across a second displacement area. The mucosal seal (or closure area) is reached at the intersection of the first and second displacement areas. FIG. 3D illustrates the competent lower esophageal sphincter 152a in the contracted position. Because the sling and clasp fibers 160a and 162a have normal force vectors, the closure area or high pressure zone formed by the sling and clasp fibers 160a and 162a has a normal length L₁ and pressure. Specifically, the resting pressure in the competent lower esophageal sphincter 152a is typically 15-25 mmHg above the intragastric pressure as measured by conventional manometry techniques. This pressure, however, can vary throughout the day. The sling and clasp fibers 160a and 162a form a competent LES 152a and accordingly maintain a normal gastroesophageal pressure gradient.

FIG. 4 is a schematic cross-sectional view of a gastroesophageal junction 150b taken generally along the line A-A of FIG. 1 in an individual 100b with a dilated cardia 150b. When the cardia 150b is chronically dilated, the oblique sling fibers 160b are separated, elongated, and angulated, modifying their length-tension properties relative to normal sling fibers 160a. These changes result in reduced LES pressure, a smaller mucosal contact area, and a shorter high pressure zone. Consequently, the LES 152b is mechanically defective.

FIGS. 5A-5D are schematic representations of the expected orientation and operation of the sling and clasp fibers 160b and 162b in the individual 100b with a dilated cardia 150b. For example, FIG. 5A illustrates the altered orientation of the sling and clasp fibers 160b and 162b at the gastroesophageal junction 150b. Specifically, the sling and clasp fibers 160b and 162b are lengthened and misaligned such that the angle of His 151b may become obtuse. FIG. 5B illustrates a force vector X₂ representing the force exerted by the individual sling fibers 160b. The force vector X₂ of the lengthened and misaligned sling fiber 160b has a horizontal component and is oriented transverse to the force vector X₁ (FIG. 3B) of the normal sling fiber 160a. FIG. 5C illustrates a combined force F₃ exerted by the individual sling fibers 160b across a third displacement area and a combined force F₄ exerted by the individual clasp fibers 162b across a fourth displacement area. The mucosal seal is reached at the intersection of the third and fourth displacement areas. The mucosal seal, however, is smaller and the high pressure zone is shortened. Thus, the LES 152b is mechanically incompetent.

FIG. 5D illustrates the incompetent lower esophageal sphincter 152b in the contracted position. The mucosal seal formed by the sling and clasp fibers 160b and 162b has a relatively short length L₂ and/or low pressure due to the altered orientation and elongation of the sling fibers 160b and/or clasp fibers 162b. Consequently, the length-tension properties of the sling and clasp fibers 160b and 162b have been altered, and the LES pressure is reduced. Because of the reduced pressure and/or short length L₂ of the mucosal seal the lower esophageal sphincter 152b is mechanically incompetent.

C. Embodiments of Methods and Apparatuses for Treating Gastroesophageal Reflux Disease

FIGS. 6-11 illustrate methods and apparatuses for treating gastroesophageal reflux disease in accordance with several embodiments of the invention. The illustrated methods recalibrate the cardia 150b by shortening the sling fibers 160b and/or clasp fibers 162b. Shortening the sling fibers 160b and/or the clasp fibers 162b reduces the perimeter of the cardia 150, thereby improving the relative relationship of the fibers 160b and 162b and allowing a more normal interplay between them, which increases LES pressure. Shortening the sling fibers 160b and/or the clasp fibers 162b also improves their length-tension properties, which also increases LES pressure. Therefore, altering the muscle fibers increases LES pressure, enlarges the mucosal seal, and lengthens the high pressure zone. As with a Nissen fundoplication, the mechanical function of the incompetent LES 152b is improved and a normal gastroesophageal pressure gradient is expected to be restored.

FIG. 6 is a schematic representation of an endoscope 170 inserted into the stomach 130b of a patient 100b with GERD in accordance with one embodiment of the invention. The illustrated endoscope 170 includes a tube 172, an energy-applying probe 180 attached at a distal portion of the tube 172, and an optical device 190 attached at the distal portion of the tube 172 near the probe 180. The tube 172 can be a flexible member and/or have a plurality of joints to properly position the probe 180 and the optical device 190 relative to a desired area of the stomach 130b and/or the gastroesophageal junction 150b. For example, the tube 172 can have a first distal section 172a for carrying the probe 180 and a second distal section 172b for carrying the optical device 190. The first and second distal sections 172a-b can move together or independently to properly position the probe 180 and the optical device 190 relative to selected sling fibers 160b and/or clasp fibers 162b (FIG. 4). The optical device 190 can be a standard endoscopic camera for providing the surgeon with a view of the tissue adjacent to the probe 180.

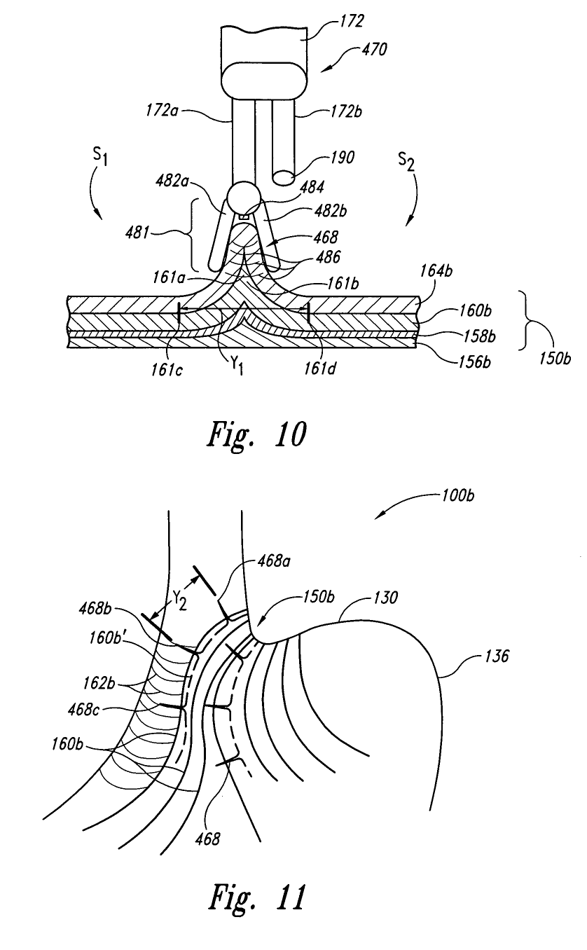

The energy-applying probe 180 can be an ultrasonic transducer, radio-frequency electrode, laser, microwave antenna, or other suitable type of probe that applies desired energy to the sling fibers 160b and/or clasp fibers 162b in order to shorten them. For example, in several embodiments, such as those described below with reference to FIGS. 7-9, the probe 180 applies ultrasonic energy to heat and shrink selected portions of the sling fibers 160b and/or clasp fibers 162b so that the length of the individual fibers 160b and/or 162b is reduced and the cardia 150b is recalibrated. In other embodiments, such as those described below with reference to FIGS. 10 and 11, the probe 180 can apply RF energy to weld sections of the individual sling fibers 160b and/or clasp fibers 162b together, thereby shortening the fibers 160b and/or 162b and recalibrating the cardia 150b. This is expected to improve the mechanical function of the LES 152b and restore the gastroesophageal pressure gradient.

FIG. 7 is a schematic representation of an endoscope 270 positioned in the cardia 150b of the stomach of a patient in accordance with another embodiment of the invention. The illustrated endoscope 270 includes a tube 172, an optical device 190 carried by the tube 172, an ultrasonic transducer 280 carried by the tube 172, and an acoustic coupler 282 adjacent to the transducer 280. The tube 172 and optical device 190 can be generally similar to the tube 172 and optical device 190 described above with reference to FIG. 6. The transducer 280 is properly positioned to apply ultrasonic energy toward a selected portion of the sling fibers 160b by placing the acoustic coupler 282 against the mucosal/submucosal layer 164b. The acoustic coupler 282 is sized to space the ultrasonic transducer 280 apart from the mucosal/submucosal layer 164b by a prescribed distance. The transducer 280 focuses ultrasonic energy in a focal zone 284 at a depth corresponding to the location of the sling fibers 160b. The transducer 280 accordingly focuses the ultrasonic energy such that the sling fibers 160b are heated to a desired temperature without damaging the mucosal/submucosal layer 164b with excessive heat.

The ultrasonic energy heats the sling fibers 160b to reduce the length of the individual fibers 160b. More specifically, heating collagenous targets in clasp and sling fibers, such as the endomysium sheath surrounding each muscle cell, shrinks the length of the individual sling and clasp fibers. Several embodiments of the invention apply ultrasonic energy in a manner that heats the collagenous targets to a temperature of approximately 50° C. to approximately 100° C. for a period of time sufficient to shrink the clasp and/or sling fibers. Additionally, the energy is preferably focused below the surface so that the mucosal/submucosal layer is much cooler than the collagenous targets and is not damaged by the ultrasonic energy. In the illustrated embodiment, the ultrasonic transducer 280 applies ultrasonic energy to shrink a section of the individual sling fibers 160b within the focal zone 284. By shrinking a section of an individual sling fiber 160b, the length of the entire sling fiber 160b is reduced. As described below with reference to FIG. 8, the transducer 280 can be scanned across the stomach 130b and/or gastroesophageal junction 150b to heat and shrink various sections of at least some of the sling fibers 160b and/or the clasp fibers 162b.

FIG. 8 illustrates one example of a pattern of heated affected zones 288 after scanning the ultrasonic transducer 280 across the stomach 130b and gastroesophageal junction 150b of a patient 100b in accordance with one embodiment of the invention. The ultrasonic transducer 280 applies ultrasonic energy to selected zones 288 of the stomach 130b and gastroesophageal junction 150b to recalibrate the cardia 150b. Specifically, the ultrasonic transducer 280 heats sections of several sling fibers 160b with each zone 288 to shrink the fibers 160b. For example, the ultrasonic transducer 280 heats and shrinks a first section 161a of a first sling fiber 160b′ within a first zone 288a, which reduces the distance between sections of the first sling fiber 160b′ on opposite sides of the first zone 288a and shortens the length of the first sling fiber 160b′. The transducer 280 heats multiple zones 288 to shrink various sections of numerous sling fibers 160b to recalibrate the cardia 150b and restore LES competency. One measure of a recalibrated cardia and restored LES might be a restoration of a normal gastroesophageal pressure gradient. To this end, the pressure in the lower esophageal sphincter 152b can be measured during treatment via conventional manometry techniques to determine when the pressure is within a normal range and the shrinkage of the sling fibers 160b is sufficient. Moreover, the stomach 130b can be distended with gas to facilitate scanning in several embodiments.

In the illustrated example, the heat affected zones 288 include segments of all of the sling fibers 160b. In other embodiments, however, the heat affected zones 288 may not include a segment of several sling fibers 160b. For example, only the sling fibers 160b closest to the clasp fibers 162b can be heated to provide a specific directional correction to the forces exerted by the fibers 160b in several methods. Moreover, although the illustrated heat affected zones 288 are oriented in a direction generally transverse to the fibers 160b to maximize shrinkage, in other embodiments, the heat affected zones 288 can have a different position relative to the sling fibers 160b. Furthermore, the transducer 280 may also heat and shrink sections of the clasp fibers 162b in lieu of or in addition to the sling fibers 160b. In either case, the transducer 280 moves across portions of the stomach 130b and/or gastroesophageal junction 152b to heat sections of the sling fibers 160b and/or clasp fibers 162b and shorten the length of the fibers 160b and/or 162b.

In an additional embodiment, the afferent nerves in the cardia 150b can be electrically stimulated to cause a transient relaxation of the sling fibers 160b and/or clasp fibers 162b before scanning the transducer 280 across the stomach 130b and/or the gastroesophageal junction 150b. It is expected that reducing the tension of the sling fibers 160b and/or clasp fibers 162b while scanning the transducer 280 will reduce the energy required to shorten the sling fibers 160b and/or clasp fibers 162b. In other embodiments, however, the afferent nerves may not be electrically stimulated.

One feature of the method described above with reference to FIGS. 7 and 8 is that the ultrasonic transducer 280 heats selected tissue to reduces the length of the individual fibers 160b and/or 162b. The shortened fibers 160b and/or 162b increase LES pressure and recalibrate the cardia, such as is accomplished with anti-reflux surgery. The altered sling fibers 160b and/or clasp fibers 162b are expected to result in a competent LES. As such, the illustrated method provides a long-term solution to GERD that does not involve many of the risks of conventional treatments.

Another advantage of the method described above with reference to FIGS. 7 and 8 is that the cardia can be recalibrated and LES pressure increased without significantly damaging the surrounding tissue. For example, the ultrasound transducer 280 focuses the ultrasonic energy so that the mucosal/submucosal layer 164b is not exposed to excessive heat. Moreover, the illustrated method does not require the implantation of clips, staples, sutures, or other objects. As such, the illustrated method is expected to be a safe and effective treatment of GERD.

D. Additional Embodiments of Methods and Apparatuses for Treating Gastroesophageal Reflux Disease

FIG. 9 is a schematic representation of an endoscope 370 in accordance with another embodiment of the invention. The illustrated endoscope 370 includes a plurality of ultrasonic transducers 380 (only four of which are shown and identified as 380a-d) and an acoustic coupler 382 adjacent to the transducers 380. The transducers 380 are arranged in an array and can be selectively operated to generate a focal zone 384 at a desired depth in the tissue. For example, selected transducers 380 can be operated such that the ultrasonic energy is amplified via constructive interference at the depth of the sling fibers 160b. This advantageously reduces the energy requirements for each transducer 380 and, consequently, the heat damage to the mucosal/submucosal layer 164b. Moreover, the transducers 380 can be electronically phased to modify the depth and size of the focal zone 384. As a result, the depth and size of the focal zone 384 can be changed by operating different combinations of transducers 380. Suitable transducers are described in U.S. Pat. Nos. 6,719,694; 6,656,136; and 6,626,855 all of which are incorporated herein by reference in their entireties.

FIG. 10 is a schematic representation of an endoscope 470 in accordance with another embodiment of the invention. The illustrated endoscope 470 includes a radio-frequency applicator 481. The illustrated radio-frequency applicator 481 includes a first grasping member 482a, a second grasping member 482b, and a suction device 484 between the first and second grasping members 482a-b. The first and second grasping members 482a-b form forceps and define a bi-polar electrode radio-frequency energy applicator for reliably securing a portion of tissue and applying radio-frequency energy to the tissue. Specifically, the suction device 484 draws the portion of tissue toward the bi-polar applicator 481 while the first grasping member 482a pivots in a first direction S₁ and the second grasping member 482b pivots in a second direction S₂ so that the members 482a-b grasp the portion of tissue. After securing the portion of tissue, the first and second grasping members 482a-b apply radio-frequency energy 486 with a predetermined intensity over a predetermined time period to form a weld 468 in the tissue. Feedback control can also be used to determine when to stop treatment. As a result, first and second sections 161a-b of the individual sling fibers 160b are welded together. In several embodiments, the weld 468 reduces a distance Y₁ between third and fourth sections 161c-d of the individual sling fibers 160b by approximately 5 mm. As described below with reference to FIG. 11, the endoscope 470 can form a plurality of welds 468 in sling fibers 160b and/or clasp fibers 162b to shorten them and re-calibrate the cardia 150b.

In other embodiments, the endoscope may have other configurations. For example, the energy applicator may apply ultrasonic, light, microwave, or other suitable types of energy. Moreover, the endoscope may further include a cooling system for cooling the applicator 481, and/or a thermocouple for monitoring the temperature of the mucosal/submucosal layer 164b. In addition, the endoscope may include a wire shaped in a helical configuration for drawing the tissue toward the energy applicator 481 in lieu of the suction device 484. Alternatively, the endoscope may not include an additional means for securing the tissue besides the bi-polar forceps.

FIG. 11 illustrates one example of a pattern of welds 468 formed in a patient 100b in accordance with one embodiment of the invention. In the illustrated embodiment, several sling fibers 160b have multiple welds 468 and other sling fibers 160b do not include any welds 468. For example, a first sling fiber 160b′ includes first, second, and third welds 468a-c, and adjacent welds 468 are separated by a distance Y₂ of approximately 10 mm. In other embodiments, however, the sling fibers 160b can have different numbers of welds 468, the spacing between adjacent welds 468 can be different, and/or welds 468 may be formed on at least some of the clasp fibers 162b in lieu of or in addition to the sling fibers 160b. In either case, the number and spacing of the welds 468 are selected to shorten the lengthened sling fibers 160b and/or clasp fibers 162b and recalibrate the cardia 150b. As a result, the altered sling fibers 160b and/or clasp fibers 162b are expected to restore the competence of the LES.

From the foregoing, it will be appreciated that specific embodiments of the invention have been described herein for purposes of illustration, but that various modifications may be made without deviating from the spirit and scope of the invention. For example, although the illustrated transducers apply ultrasonic or radio-frequency energy, other transducers can apply microwave, light, and/or other types of energy. Accordingly, the invention is not limited except as by the appended claims.

I/We claim:

1. A method of treating an esophageal disorder in a patient, the method comprising applying energy to a target zone in at least one of (a) the gastric sling fibers at the cardia and/or the stomach, or (b) the semicircular clasp fibers at the esophagus and/or the cardia to improve the function of the lower esophageal sphincter.

2. The method of claim 1 wherein applying energy to the target zone comprises applying ultrasonic energy toward the target zone.

3. The method of claim 1 wherein applying energy to the target zone comprises applying radio-frequency energy toward the target zone.

4. The method of claim 1 wherein:
the individual fibers in the target zone comprise a first section and a second section spaced apart from the first section; and
applying energy to the target zone comprises reducing a distance between the first and second sections.

5. The method of claim 1 wherein applying energy to the target zone comprises heating the target zone to shorten the individual fibers in the target zone.

6. The method of claim 1 wherein:
the individual fibers in the target zone comprise a first section and a second section spaced apart from the first section; and
applying energy to the target zone comprises welding together the first and second sections of at least some of the individual fibers in the target zone.

7. The method of claim 1, further comprising inserting an endoscope with an energy-applying probe into the patient, wherein applying energy to the target zone comprises applying energy from the probe to the target zone.

8. The method of claim 1 wherein applying energy to the target zone comprises applying energy to the target zone to increase the resting pressure of the lower esophageal sphincter.

9. The method of claim 1 wherein applying energy to the target zone comprises applying energy to the target zone to lengthen the high pressure zone in the lower esophageal sphincter.

10. The method of claim 1, further comprising measuring a pressure in the lower esophageal sphincter, and wherein applying energy to the target zone comprises applying energy to the target zone until reaching a predetermined pressure in the lower esophageal sphincter.

11. The method of claim 1, further comprising electrically stimulating the afferent nerves in the patient to cause a transient relaxation in at least one of the sling fibers and the clasp fibers, and wherein applying energy to the target zone comprises applying energy to the target zone while at least one of the sling fibers and the clasp fibers is relaxed.

12. The method of claim 1 wherein applying energy comprises applying energy to the target zone without significantly injuring adjacent tissue.

13. A method of treating an esophageal disorder in a patient, the method comprising:
inserting a probe for applying energy into the patient; and
applying energy from the probe to at least one of (a) a sling fiber at the cardia and/or the stomach, or (b) a clasp fiber at the esophagus and/or the cardia of the patient to reduce a distance between first and second sections of the at least one sling fiber or clasp fiber.

14. The method of claim 13 wherein applying energy from the probe comprises applying energy to increase the pressure of the lower esophageal sphincter.

15. The method of claim 13 wherein applying energy from the probe comprises applying energy to lengthen the high pressure zone in the lower esophageal sphincter.

16. The method of claim 13 wherein applying energy from the probe comprises welding together third and fourth sections of the at least one sling fiber or clasp fiber.

17. The method of claim 13 wherein applying energy from the probe comprises shortening the at least one sling fiber or clasp fiber.

18. The method of claim 13 wherein applying energy from the probe comprises applying ultrasonic energy to the at least one sling fiber or clasp fiber.

19. The method of claim 13 wherein applying energy from the probe comprises applying radio-frequency energy to the at least one sling fiber or clasp fiber.

20. A method of treating an esophageal disorder in a patient, the method comprising:
inserting a probe into the patient; and
applying energy from the probe toward at least one of (a) the sling fibers at the stomach, (b) the clasp fibers at the stomach, or (c) the gastroesophageal junction of the patient to shorten at least one of the sling or clasp fibers.

21. The method of claim 20 wherein applying energy from the probe comprises heating the at least one sling fibers at the stomach, clasp fibers at the stomach, or gastroesophageal junction.

22. The method of claim 20 wherein applying energy from the probe comprises applying ultrasonic energy to the at least one sling fibers at the stomach, clasp fibers at the stomach, or gastroesophageal junction.

23. The method of claim 20 wherein applying energy from the probe comprises applying energy to increase the resting pressure of the lower esophageal sphincter.

24. The method of claim 20 wherein applying energy from the probe comprises applying energy to lengthen the high pressure zone of the lower esophageal sphincter.

25. The method of claim 20 wherein:
the probe includes a plurality of transducers arranged in an array; and
the method further comprises applying energy from the plurality of transducers toward the at least one sling fibers at the stomach, clasp fibers at the stomach, or gastroesophageal junction.

26. The method of claim 20 wherein:
the probe includes a plurality of transducers arranged in an array; and
applying energy comprises focusing the depth of the energy transmitted from at least some of the transducers toward the at least one sling fibers at the stomach, clasp fibers at the stomach, or gastroesophageal junction.

27. The method of claim 20 wherein:
the probe includes a plurality of transducers arranged in an array; and
applying energy from the plurality of transducers comprises phasing the energy from at least some of the transducers.

28. The method of claim 20 wherein applying energy comprises applying energy to a plurality of spaced-apart target zones at selected locations on the stomach and/or gastroesophageal junction to calibrate the cardia.

29. A method of treating an esophageal disorder in a patient, the method comprising applying ultrasonic energy to a target zone in at least one of the sling fibers at the stomach, the clasp fibers at the stomach, and the gastroesophageal junction of the patient to shorten the length of at least one of the sling fibers and the clasp fibers and increase the resting pressure in the lower esophageal sphincter.

30. The method of claim 29 wherein applying ultrasonic energy comprises applying ultrasonic energy from a plurality of ultrasonic transducers arranged in an array.

31. The method of claim 29 wherein applying ultrasonic energy from the transducers comprises applying ultrasonic energy to the target zone to lengthen the high pressure zone of the lower esophageal sphincter.

32. The method of claim 29 wherein applying ultrasonic energy comprises focusing the depth of the ultrasonic energy applied to the target zone.

33. The method of claim 29 wherein applying ultrasonic energy comprises:
applying ultrasonic energy from a plurality of transducers arranged in an array; and
phasing the applied ultrasonic energy from at least some of the transducers.

34. A method of treating an esophageal disorder in a patient, the method comprising welding first and second sections of at least some of the individual sling and/or clasp fibers at the stomach and/or gastroesophageal junction of the patient by directing energy toward the at least some sling and/or clasp fibers to improve the mechanical function of the lower esophageal sphincter by lengthening the high pressure zone.

35. The method of claim 34 wherein welding the first and second sections of at least some of the individual sling and/or clasp fibers comprises applying radio-frequency energy to the first and second sections of the at least some individual sling and/or clasp fibers.

36. The method of claim 34 wherein welding the first and second sections of at least some of the individual sling and/or clasp fibers comprises joining the first and second sections of the at least some individual sling and/or clasp fibers without suturing and/or stapling the tissue in the stomach and/or gastroesophageal junction.

37. The method of claim 34 wherein welding the first and second sections of at least some of the individual sling and/or clasp fibers comprises grasping tissue having the first and second sections of the at least some individual sling and/or clasp fibers with forceps.

38. The method of claim 34 wherein welding the first and second sections of at least some of the individual sling and/or clasp fibers comprises:
securing tissue having the first and second sections of the at least some individual sling and/or clasp fibers with first and second grasping members; and
applying radio-frequency energy from the first and/or second grasping member to the at least some sling and/or clasp fibers.

39. The method of claim 34 wherein welding the first and second sections of at least some of the individual sling and/or clasp fibers comprises drawing tissue having the first and second sections of the at least some individual sling and/or clasp fibers with a suction device.

40. The method of claim 34 wherein welding the first and second sections of at least some of the individual sling and/or clasp fibers comprises drawing tissue having the first and second sections of the at least some individual sling and/or clasp fibers with a wire shaped in a helical configuration.

41. A method of treating an esophageal disorder in a patient, the method comprising:
inserting a probe into the patient; and
applying energy from the probe toward at least a portion of the sling and/or clasp fibers at the stomach and/or gastroesophageal junction of the patient to form welds in at least some of the individual sling and/or clasp fibers and reduce a distance between first and second sections of the at least some individual sling and/or clasp fibers.

42. The method of claim 41 wherein applying energy comprises applying radio-frequency energy to at least a portion of the sling and/or clasp fibers.

43. The method of claim 41 wherein applying energy comprises forming welds in the at least some individual sling and/or clasp fibers without suturing and/or stapling the tissue in the stomach and/or gastroesophageal junction.

44. The method of claim 41, further comprising securing tissue having the at least some individual sling and/or clasp fibers with first and second grasping members, wherein applying energy comprises applying radio-frequency energy from the first and/or second grasping member to the at least some sling and/or clasp fibers.

45. A method of treating an esophageal disorder in a patient, the method comprising:
inserting a probe into the patient; and
a step for improving the mechanical function of the lower esophageal sphincter in the patient.

46. The method of claim 45 wherein the step for improving the mechanical function of the lower esophageal sphincter comprises applying energy to at least some of the sling and/or clasp fibers at the stomach and/or gastroesophageal junction.

47. The method of claim 45 wherein the step for improving the mechanical function of the lower esophageal sphincter comprises applying ultrasonic energy to at least some of the sling and/or clasp fibers at the stomach and/or gastroesophageal junction.

48. The method of claim 45 wherein the step for improving the mechanical function of the lower esophageal sphincter comprises applying radio-frequency energy to at least some of the sling and/or clasp fibers at the stomach and/or gastroesophageal junction.

49. The method of claim 45 wherein the step for improving the mechanical function of the lower esophageal sphincter comprises welding first and second sections of at least some of the individual sling and/or clasp fibers in the stomach and/or gastroesophageal junction.

50. The method of claim 45 wherein the step for improving the mechanical function of the lower esophageal sphincter comprises reducing a distance between first and second sections of at least some of the individual sling and/or clasp fibers at the stomach and/or gastroesophageal junction.

51. An apparatus for treating an esophageal disorder in a patient, the apparatus comprising:
an endoscope for insertion into the patient; and
an applicator coupled to the endoscope for applying energy to at least one of (a) the sling fibers at the stomach, (b) the clasp fibers at the stomach, or (c) the gastroesophageal junction of the patient wherein the applicator is configured to releasably grasp a portion of tissue containing sections of at least one of the sling fibers or the clasp fibers.

52. The apparatus of claim 51 wherein the applicator comprises first and second grasping members movable relative to each other between (a) a first position in which the first and second grasping members grasp the portion of tissue, and (b) a second position in which the first and second grasping members release the portion of tissue.

53. The apparatus of claim 51, further comprising an optical device coupled to the endoscope and positioned proximate to the applicator.

54. The apparatus of claim 51, further comprising a device for drawing the portion of tissue toward the applicator.

55. The apparatus of claim 51 wherein the applicator comprises one or more radio-frequency electrodes.

56. The apparatus of claim 51 wherein the applicator comprises forceps.

57. An apparatus for treating an esophageal disorder in a patient, the apparatus comprising:
an endoscope for insertion into the patient;
means for applying energy to at least one of the sling fibers or the clasp fibers in the patient; and
means for releasably securing a portion of tissue containing sections of at least one of the sling fibers or the clasp fibers.

58. The apparatus of claim 57 wherein the means for releasably securing comprise forceps.

59. The apparatus of claim 57 wherein the means for releasably securing comprise first and second members movable relative to each other between (a) a first position in which the first and second members secure the portion of tissue, and (b) a second position in which the first and second members release the portion of tissue.

60. The apparatus of claim 57 wherein the means for applying energy comprise one or more radio-frequency electrodes.

61. The apparatus of claim 57 wherein the means for applying energy comprise one or more ultrasonic transducers.

62. An apparatus for treating an esophageal disorder in a patient, the apparatus comprising:
an endoscope for insertion into the patient;
a securing device coupled to the endoscope, the securing device configured to releasably secure a portion of tissue containing sections of at least one of the sling fibers or the clasp fibers; and
an energy applicator coupled to the endoscope and positioned for applying energy to the portion of tissue secured by the securing device.

63. The apparatus of claim 62 wherein the energy applicator comprises one or more radio-frequency electrodes.

64. The apparatus of claim 62 wherein the securing device comprises forceps.

65. The apparatus of claim 62 wherein the securing device comprises first and second members movable relative to each other between (a) a first position in which the first and second members secure the portion of tissue, and (b) a second position in which the first and second members release the portion of tissue.
